(19) United States
(12) Patent Application Publication
(10) Pub. No.: US 2001/0034479 A1
(43) Pub. Date: Oct. 25, 2001

(54) Optically based transcutaneous blood gas sensor

(76) Inventors: Lawrence S. Ring, Laguna Beach, CA (US); Paul D. Levin, Santa Cruz, CA (US)

Correspondence Address: Bruce H. Johnsonbaugh, Eckhoff, Hoppe, Slick, Mitchell & Anderson, Suite 760, Four Embarcadero Center, San Francisco, CA, 94111, US

(21) Appl. No.: 09/754,177

(22) Filed: Jan. 4, 2001

Related U.S. Application Data
(63) Continuation-in-part of application No. 09/553,439, filed on Apr. 19, 2000.

(51) Int. Cl. A61B 5/00; G01N 21/17

(52) U.S. Cl. 600/322; 600/310; 422/82.05; 600/312

(57) ABSTRACT

An optically based transcutaneous blood gas sensor is provided having a reusable sensor head and a disposable sensing cap. A separate receptacle detachably connects to the sensor head and is attached to a person's skin by a layer of adhesive after a release liner is removed. The sensor head includes a sealed cavity housing one or more LEDs and one or more photodetectors. The disposable cap carries fluorescent targets which produces an amount of fluorescence as a function of blood gas concentration and the fluorescence is sensed by the photodetectors.

### CROSS-REFERENCE TO RELATED APPLICATION

This is a continuation-in-part of U.S. patent application Ser. No. 09/553,439 filed Apr. 19, 2000.

### FIELD OF THE INVENTION

Transcutaneous monitoring of Oxygen (O₂) and Carbon Dioxide (CO₂) has been available since the early 1980s and has been found to be especially useful in the neonatal intensive care unit. The Transcutaneous monitors presently available measure oxygen with a Clark polarographic electrode, and CO₂ with a Severinghaus potentiometric electrode. The latter is basically a standard glass pH measuring electrode bathed in a bicarbonate solution, the pH of which changes in response to the diffusion of CO₂ into the electrolyte solution from the skin. The present invention substitutes an optical system using fluorescent quenching and utilizes a reusable sensor head and a disposable cap which holds fluorescent targets in place over the sensor head.

### BACKGROUND OF THE INVENTION

Much effort has been spent in improving these basic techniques to give a device which is durable, accurate, and convenient for the user. For example, Danek in U.S. Pat. No. 4,303,076 developed a housing for the electrode with a cap to hold the gas permeable membrane which was assembled with a snap-fitting ring. Pedersen in U.S. Pat. No. 4,836,907 describes a double membrane system for simultaneously measuring partial pressure of both oxygen and CO₂. Vesterager and Jeppesen in U.S. Pat. No. 4,274,418 describe an open-ended annular mounting ring which fastens to the skin of a patient allowing the sensor itself to be releasably attached to the patient. The same patent describes a mounting tool for conveniently mounting the double membrane system onto the sensor. A change from glass to ceramic pH electrodes by several companies has made Transcutaneous monitoring more durable and has greatly increased the longevity of this type of sensor.

In spite of these improvements, there still remain several disadvantages to Transcutaneous monitors using electrochemical sensors. The Clark-type polarographic electrode consumes oxygen as it measures the partial pressure of the gas. To maintain accuracy with this type of sensor a steady flow of tissue oxygen in excess of this consumption is required. This is generally accomplished by incorporating a skin heater in the electrode to heat the skin. A temperature of 44 degrees causes vasodilatation of the arterioles under the sensor. Due to the high temperature of the heating element, the sensor position on the skin must be changed every few hours to avoid burns. A recalibration is usually required each time that the electrode is moved.

Drift of both the CO₂ electrode and the oxygen polarographic electrode is difficult to eliminate in instruments with electrochemical sensors. As a result, conventional Transcutaneous monitors usually require recalibration several times a day. Finally, the cost of the complex Transcutaneous sensors is quite high due to the complicated mechanism and the necessity for miniaturizing many of the small parts within the standard electrochemical sensor.

The present invention largely solves the above-mentioned problems by using an optically based rather than an electrochemically based sensor. Light emitting diodes (LEDs) are used to direct light of the appropriate wave lengths against a fluorescent target on the surface of the skin, and the amount of fluorescence produced is a function of the oxygen or carbon dioxide partial pressure.

The use of fluorescence quenching rather than electrochemical sensing greatly reduces the cost of the sensing unit and the accompanying electronics and largely eliminates the internal drift of the sensing system. Since oxygen is not consumed in this method a lower oxygen flow is permissible and therefore the sensor's heater can be run cooler than when using a conventional oxygen electrode.

The possibility of using fluorescence quenching for measuring oxygen transcutaneously was suggested by Fostick in U.S. Pat. No. 4,041,932. This patent taught the use of a small chamber sealed to the skin. Oxygen was to be measured by directing ultraviolet light against a fluorescent target within the chamber through a fiber optic cable, the light source being outside the chamber. Fluorescence from the target was measured with a second fiber optic bundle to an outside photo detector. Fostick further discloses in this patent the use of a disposable plastic member sealed to the skin for the direct measurement of CO₂ by infrared light absorption.

A type of optical sensor for measuring CO₂ transcutaneously was described by Ullrich in U.S. Pat. No. 4,930,506. A color indicator bound to a thin layer of acrylic resin was placed between a light emitting diode and a light detector with all of these elements being placed on the same outer surface of the permanent sensor. A snap on ring/membrane device was to be clipped onto the combined sensor which was also designed to measure oxygen saturation by the use of reflection pulse oximetry. In the Ullrich device, the membrane simply held an electrolyte solution against the face of the sensor and was not itself the target of light from the LED.

U.S. Pat. No. 5,342,789 by Chick describes the use of certain fluorescent chemistries placed “in, on, or under the skin” for the measurement of glucose. No examples or other explanation are provided for placement “on the skin”. The teaching is oriented toward the direct interface of the chemistries with internal body fluids and one can presume that the intent of an “on skin” sensor was in conjunction with means for extracting these fluids to the sensor.

U.S. Pat. No. 4,401,122 by Clark similarly describes the implantation of calorimetric agents under the skin for the measurement of various constituents of biological interest.

U.S. Pat. No. 5,508,509 by Yafuso describes techniques for the manufacture of fluorescent sensing elements. These elements are disclosed for use in a disposable “cassette” system external to the body. The use of these elements in the measurement of analytes in an aqueous medium such as blood is disclosed.

P. E. M. Huygen, et. al. describes an LED based sensor device for respiratory monitoring in “An In-Line Oxygen Gas-Fraction Sensor for Anesthesia and Intensive Care”, Oxygen Transport to Tissue XIX, Plenum Press, 1997. This article describes some of the drift and stability issues affecting these type of devices.

D. W. Lubbers, et. al. describes the use of a fluorescent membrane applied to the skin for oxygen measurement in “O2 Flux Optode”, Oxygen Transport to Tissue XVII, Plenum Press, 1996. This system uses fiber optics for interfacing to the film. Later references use video imaging for area wide monitoring.

### SUMMARY OF THE INVENTION

This invention relates to a sensor using optical rather than electrical chemical components to measure transcutaneous gases. The sensor has three main components, a reusable sensor head, a disposable cap for the sensor which holds a fluorescent target layer excited by light from the reusable sensor head, and a support receptacle which holds the first two components in place against the patient's skin. The sensor head is bell-shaped, approximately ⅝″ high and ¾″ in diameter, and covered with a plastic jacket. It is connected by a multistrand cable to a monitor containing electrical components, operator controls, and a display. The optical sensor head and its cable are intended for repeated use with many patients. The sensor cable can be disconnected from the monitor for sterilization if desired.

Fluorescent target layers are applied to the underside of the transparent sensor cap. The targets are two 3 mm discs containing fluorescent material sensitive to oxygen and CO₂. The fluorescent targets are bonded to the underside of the disposable sensor cap and are covered with a gas permeable white opaquing layer which increases the detection signal strength by reflecting fluorescent light back to the sensor. The overcoat layer is hygroscopic and helps to retain moisture around the targets, which is particularly important for the accurate detection of CO₂.

The reusable sensor head and disposable sensor cap are held against the skin by a disposable receptacle. An encircling flange of the receptacle carries a sticky layer which is covered with protective paper until just prior to applying the receptacle to the patient's skin. Removal of the protective paper cover exposes the sticky surface on the underside of the flange. The receptacle is similar to that described in U.S. Pat. No. 4,274,418 assigned to Radiometer.

The electronics of the reusable sensor head are all hermetically sealed within a “TO” type can. The preferred embodiment uses a TO-8 can approximately 0.3″ high by 0.6″ in diameter. Within the TO-8 can are 430 and 470 nanometer LEDs, along with reference and signal detecting photodiodes and other environmentally sensitive optoelectronic components.

Light is emitted from lensed LEDs at a narrow angle. This characteristic of the LEDs enables separate spots of fluorescent material, sensitive to oxygen and carbon dioxide to be located on the target layer a few millimeters apart without interfering with each other. The LEDs are flashed sequentially several times per second.

LED#1 (430 nm) and LED #2 (470 nm) are both filtered to block light above 480 nm. The emitted light from each LED strikes its corresponding target layer dye spot causing an emission to occur. The emission light travels outward in all directions with a portion reaching each of the large photodetectors after passing through their respective emission filters. The intensity of the detected light is interpreted by software to give the partial pressure of oxygen or carbon dioxide.

A flat heating element on the face of the TO-8 can is provided to raise the underlying skin temperature to as high as needed, depending on the age of the patient and the parameter to be measured. The heating element is configured with a clear central area to allow for the passage of light.

The dye spot differs depending on whether it is to monitor the partial pressure of oxygen or the partial pressure of CO₂. When monitoring oxygen, the dye spot is made with an embedded fluorescent dye sensitive to the quenching effects of oxygen. When the analyte to be measured is CO₂, the silicone has dispersed within it micelles of a buffer solution containing a pH responsive fluorophor. Each analyte responsive fluorescent dye spot is located within the illumination area of a discrete LED or LED pair for measurement.

Tests of the present invention have demonstrated accuracy equal to or superior to that of conventional transcutaneous sensors for both O₂ and CO₂. Response is rapid with 90 percent of full change in the fluorescent signal occurring within 60 seconds of a change in gas level. Drift levels are typically 0.1% per day which permits less frequent recalibration as compared to conventional electrochemical sensors. Additional features and advantages of this device will be enumerated in the following paragraphs.

### BRIEF DESCRIPTION OF THE DRAWINGS

FIG. 1 is an overall exploded view of the reusable sensor head and the disposable sensor support receptacle and transparent sensor cap.

FIG. 2 is an exploded sectional view of the reusable sensor head, disposable sensor support receptacle and transparent sensor cap of FIG. 1.

FIG. 3 is a sectional view of the assembled device, showing the components of FIGS. 1 and 2 assembled and ready for use.

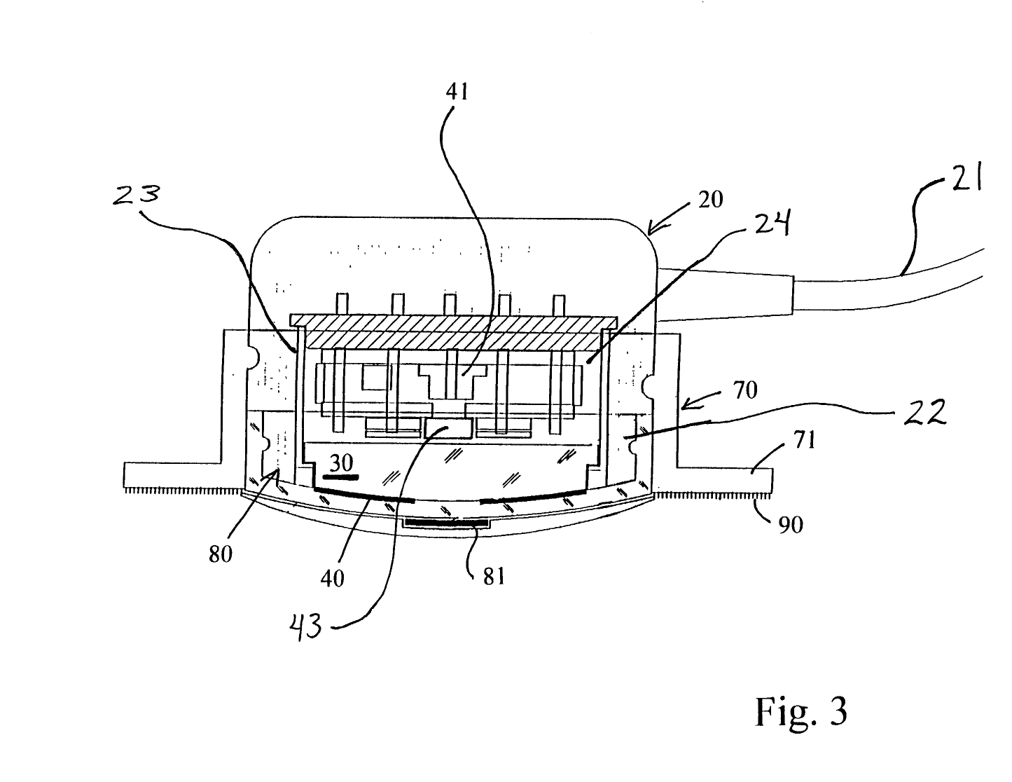

FIG. 4 is a sectional view of the assembled device of FIG. 3. The path of excitation light from the LEDs to the target layer is illustrated with arrows.

FIG. 5 is a sectional view of the assembled device of FIGS. 3 and 4. The path of emission light from the target layer to the photodetectors is illustrated with arrows.

FIG. 6 is a bottom view of the reusable sensor head.

FIG. 7 is a sectional view of the reusable sensor head of FIG. 6.

FIG. 8 is a schematic representation of the top circuit board used in the sensor head shown in FIGS. 6 and 7.

FIG. 9 is a schematic representation of the bottom circuit board used in the sensor head shown in FIGS. 6 and 7.

FIG. 10 is a frontal view of the heating element showing the central area for passage of light.

FIG. 11 is a perspective view showing the reusable sensor head of FIG. 1 assembled together with the disposable receptacle.

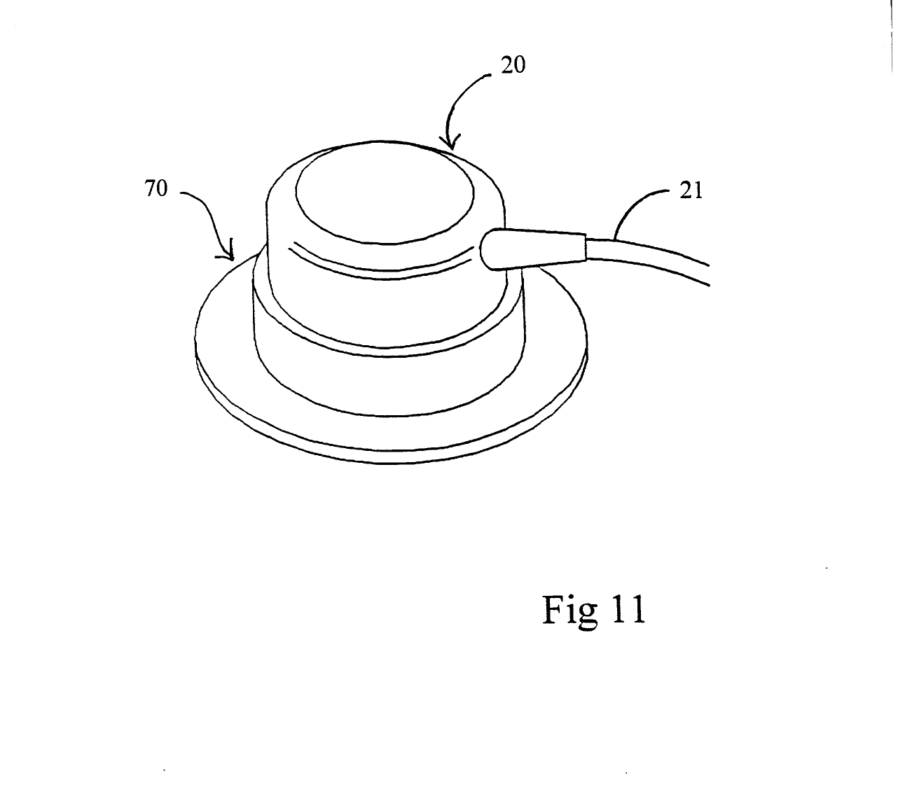

### DETAILED DESCRIPTION OF THE INVENTION

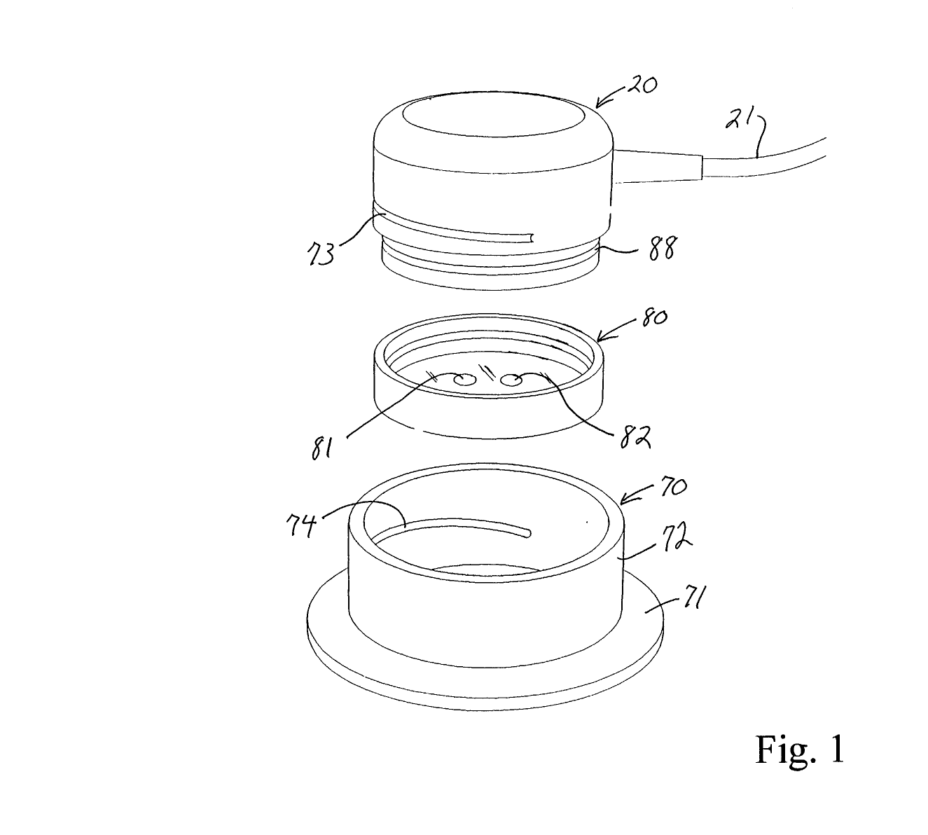

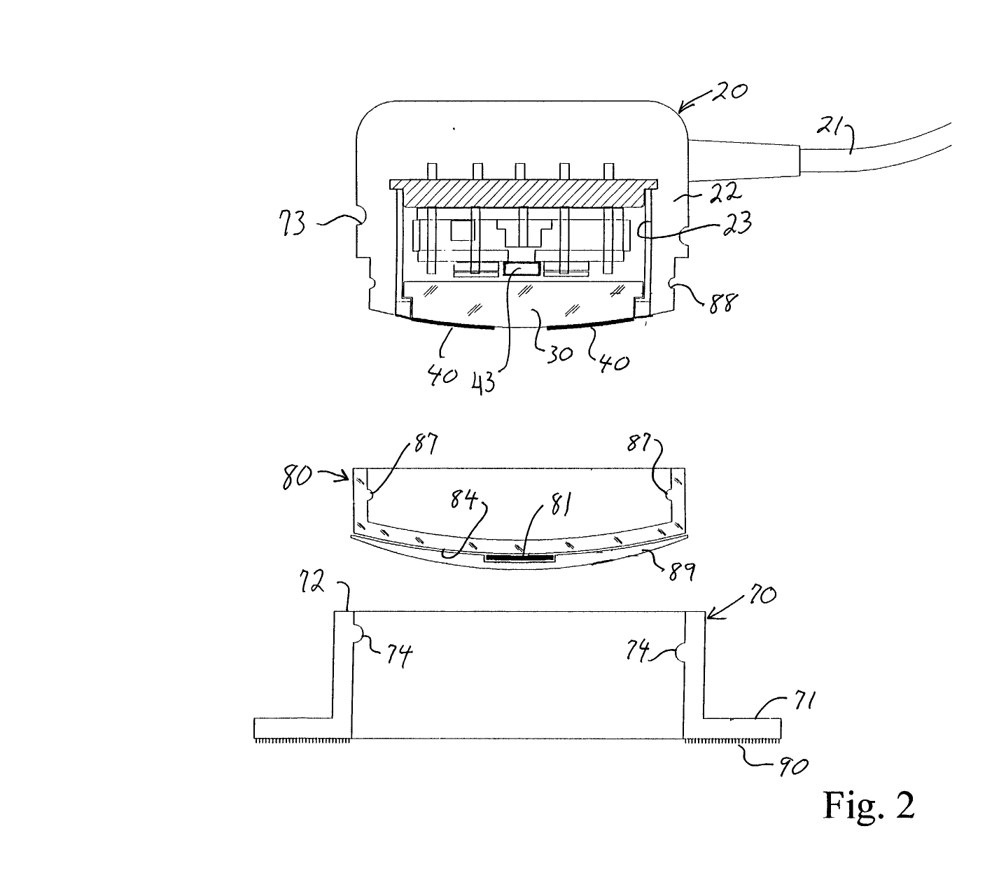

FIGS. 1 and 2 show in exploded fashion the three main parts that together make up the complete device. The upper component is the reusable sensor head 20 and its attendant cable 21. The middle component is a transparent sensor cap 80 which carries fluorescent targets 81 and 82 on its underside. The lower component is a disposable support means or receptacle 70. Receptacle 70 includes cylinder 72 with an internal spiral thread 74 which engages groove 73 formed in sensor head 20. A disc-shaped flange 71 is connected to or formed with the bottom of cylinder 72 to support the assembled device on the skin of the patient. Fluorescent targets 81 and 82 are sensitive to the effects of O₂ and/or CO₂. A ring 90 of polyurethane or similar double-sided adhesive film is adhered to the lower surface of the flange 71. The adhesive film 90 is designed to securely fasten the disposable receptacle or support means 70 to the patient's skin after removal of a protective release liner (not shown).

The transparent, disposable sensor cap 80 is optically clear but gas impermeable, and preferably transparent acrylic or polycarbonate. It serves to isolate the target layer analyte from the external environment so that only gas diffusing from the skin beneath the sensor can reach the target layer once installed.

The formulation of fluorescent targets 81 and 82 varies depending on the analyte to be monitored. Typical formulations are described in detail in many of the prior art patents discussed above including U.S. Pat. Nos. 5,508,509; 5,342,789 and 4,930,506. Preferred formulations are described in Target Layer Fabrication below. The illustration shown is for a multiple analyte target disc including first target 81 for oxygen and second target 82 for carbon dioxide.

The fluorescent targets 81 and 82 are attached directly to the lower surface 84 of sensor cap 80 with a suitable optically clear non-fluorescent adhesive such as an RTV silicone, acrylic transfer adhesive, or the like. Ultrasonic bonding or thermal staking may also be used to attach the target substrate disc to the lower surface 84 of sensor cap 80. Covering layer 89 extends over the lower surface 84. Covering layer 89 is hydroscopic and is a gas permeable white opaquing material.

The reusable sensor head 20 has a cylindrical cavity 23 containing a hermetically sealed TO-8 can 24 with a window at one end and pins for a small circuit board at its opposite end. Within the sealed can are all environmentally sensitive optoelectronic components such as LED's detectors, capacitors and filters. At the bottom of housing 22 is the glass window face or window means 30 of the TO-8 can through which the optical illumination and signal reception occur. Attached to the window 30 by epoxy or other adhesive is a slotted heater 40. Within the slot is an optional additional glass insert to provide an easy to clean optical surface for receptacle interface. The electrical connections between the reusable sensor head 20 and the primary electronics module are made by means of the flexible cord 21 with strain relief shown.

In the preferred embodiment, the window 30 is given a convex shape to facilitate displacement of air when attaching the reusable sensor head 20 to the sensor cap 80.

Transparent sensor cap 80 is disposable. It snaps onto sensor head 20 by ring 87 inside sensor cap 80 engaging a groove 88 formed on the outer surface of sensor head 20. Receptacle 70 is placed over sensor cap 80 and threadably engages sensor head 20 by thread 74 engaging groove 73 formed in sensor head 20. Sensor cap 80 and receptacle 70 are disposed of after their use with a specific patient; sensor head 20 is reused with multiple patients.

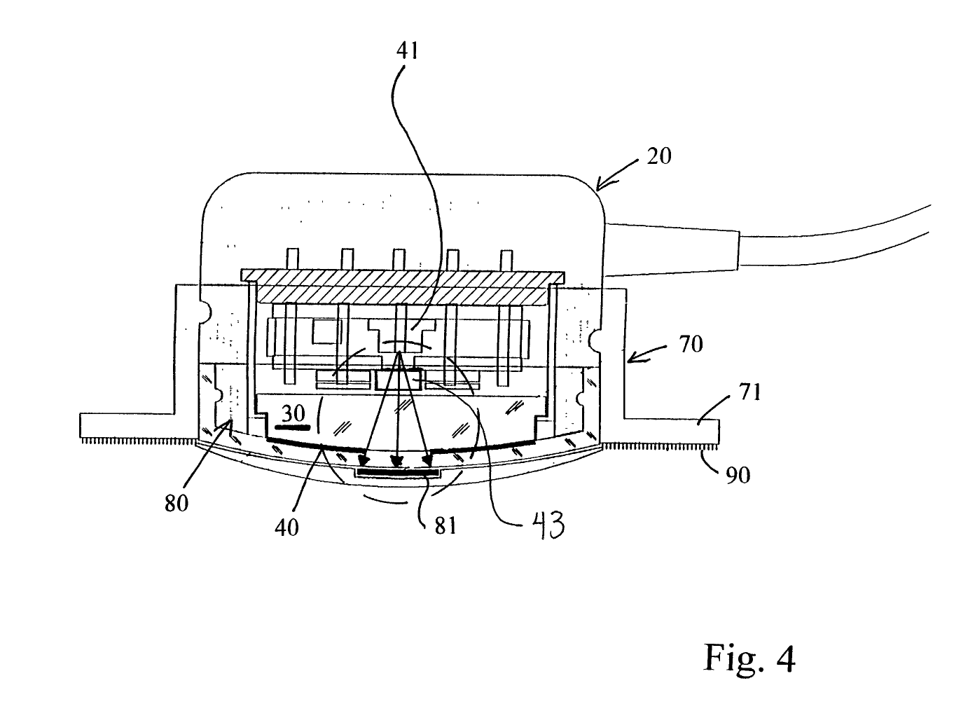

FIG. 4 shows the assembly of the reusable sensor head 20 to the receptacle or support means 70. Arrows have been overlaid to illustrate the light path for exciting light from the LEDs 41 and 42, through the excitation filter 43 and windows, and then into the target layer dye pockets 81 and 82.

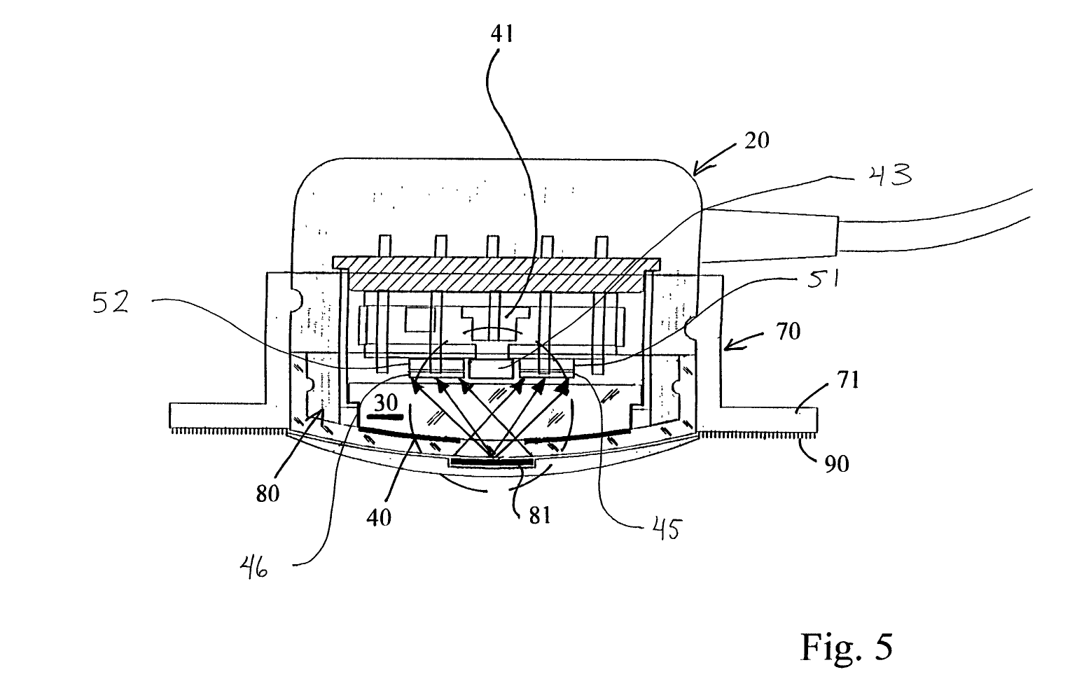

FIG. 5 also shows the assembly of the reusable sensor head 20 to the receptacle or support means 70. Arrows have been overlaid to illustrate the light path for emission light from the target layers 81 and 82, through the windows and emission filters 45 and 46, and then into the photodetectors 51 and 52.

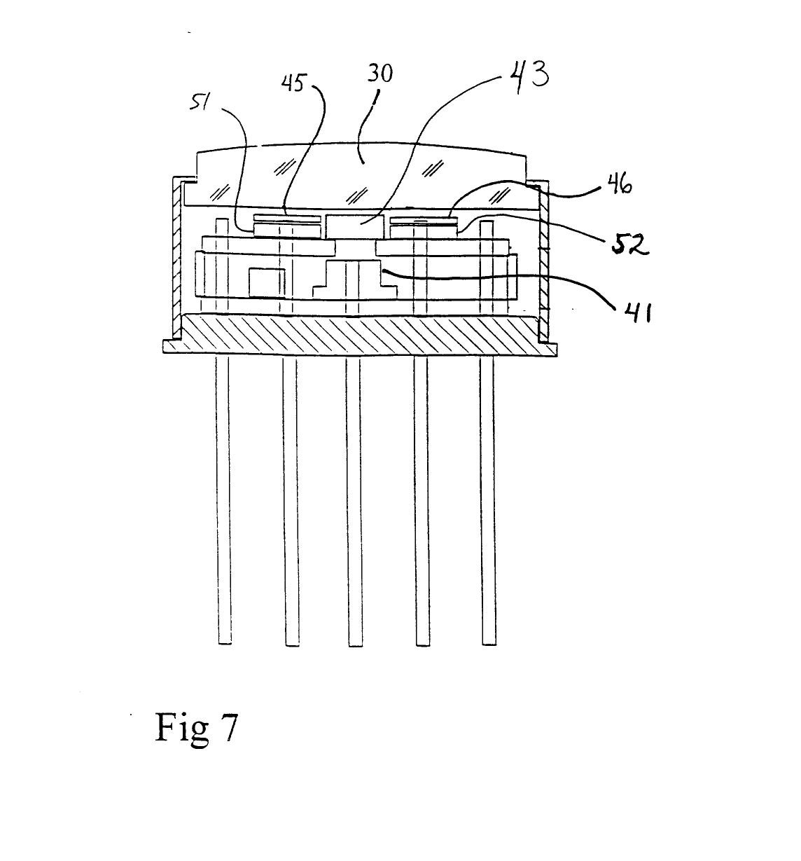

FIGS. 7, 8 and 9 are views of the TO-8 sensor can assembly and circuit boards. In developing these optical chemical sensors it was discovered that environmental sensitivity of the optical and electronic components was a significant source of drift. The TO-8 configuration illustrated here is one of many variations possible to obtain a hermetically sealed self contained fluorescence optical sensor system. The hermetically sealed can configuration isolates the optical filters from humidity, dust, debris, cleaning solvents and the like which can affect their transmission properties. It also isolates the bulk of the optical system from external mechanical stresses, isolates the high gain feedback resistors and capacitors from humidity and dust which can affect their values and noise, and isolates the photodetectors from humidity which can affect their noise level.

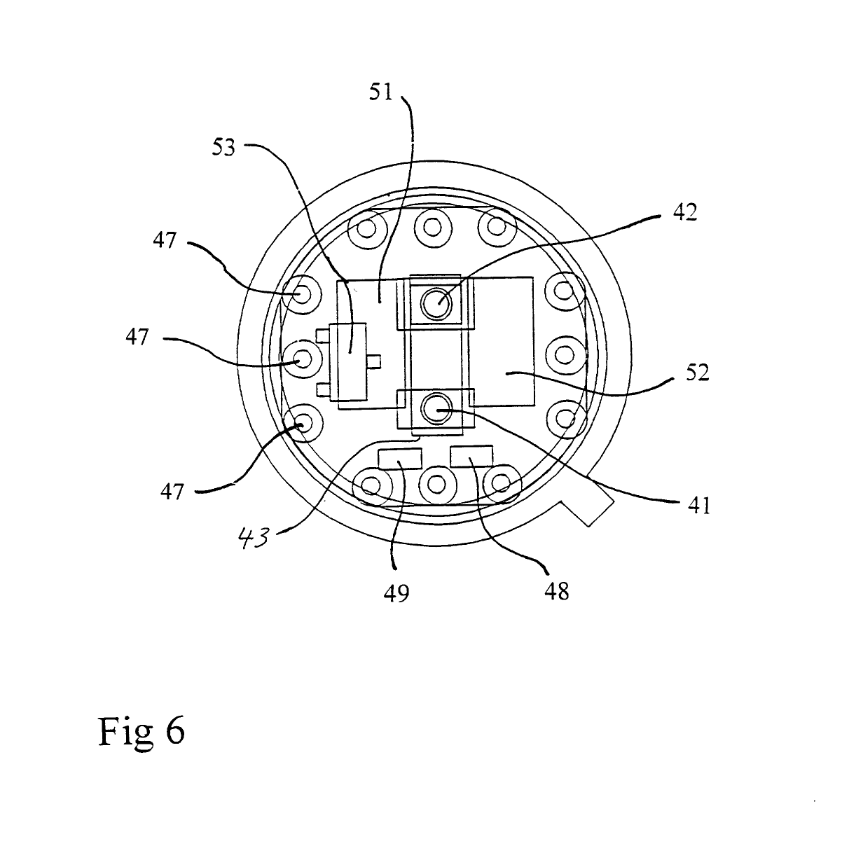

FIG. 6 is a top view of the TO-8 assembly showing the positions of the connection pins 47, reference photodetector 53, signal photodetectors 51 and 52, LEDs 41 and 42, and feedback resistor 48 and capacitor 49.

FIG. 7 is a left side cross-sectional view of the view of the TO-8 sensor window can. Each TO-8 assembly, also called a hybrid, contains the following components in order roughly from the top down:

Window can—0.6″ diameter TO-8 Kovar steel with clear optical glass (Sinclair Manufacturing Co. W600-250)

Excitation Optical filter—1 ea. 460 nm Short pass (Barr Associates custom fabricated thin film)

Emission Optical filters—#12 gel filter (Lee Filters) bonded with Lens Bond F65 (Summers Optical)

Photodetectors—0.1″×0.2″ Silicon photodetector (Photonic Detectors Inc. #PDB-C605)

Ceramic Substrate—Opaque black ceramic with laser drilled holes (Coors Ceramics ADOS-90R)

Light Emitting Diodes—Surface mount LEDs: 1 ea. 430 nm (Chicago Miniature Lamp CMD11-21UBC) and 1 ea. 470 nm (Nichia Chemical Industries NSCB100)

Photodetector—SOT-23 packaged photodetector (Photonic Detectors Inc. #PDB-C150SM)

Resistor—1M ceramic chip 0402 package (Panasonic ERJ-2RKF1.00M)

Capacitor—5 pf ceramic chip 0402 package (Panasonic ECU-E1 H050CCQ)

Ceramic Substrate—Opaque black ceramic (Coors Ceramics ADOS-90R)

Header—12 pin 0.6″ diameter 0.15″ internal pin, Kovar with ceramic pin seals (Schott SL08.138.018)

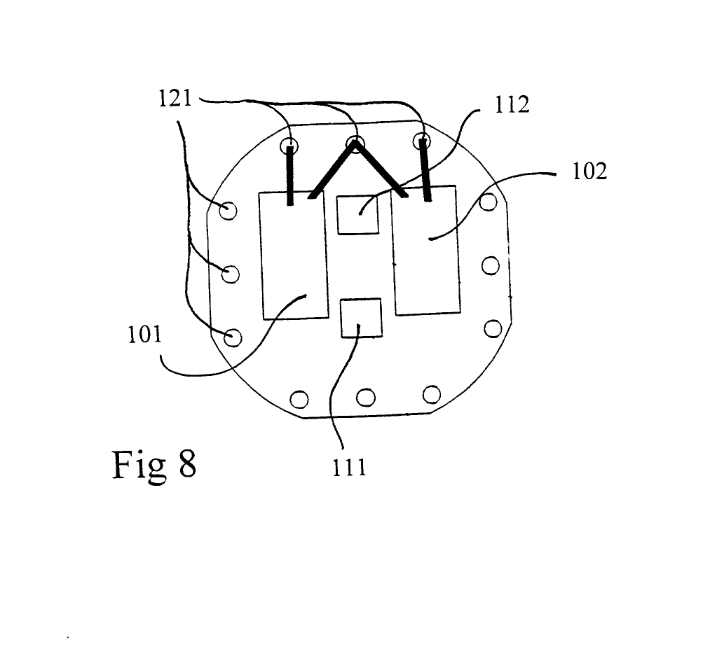

FIG. 8 shows the top circuit board with pads 101 and 102 for the signal photodetectors, square openings 111 and 112 for the LEDs to shine through from the bottom circuit board, holes 121 for the mounting pins, and circuit trace designations. The 460 nm SP excitation filter (not shown in this figure) is mounted directly over the openings to further refine the spectrum produced by the LEDs. A suitable optical epoxy such as Lens Bond F65 (Summers Optical) is used.

The emission filters are mounted to the 0.1″×0.2″ photodetectors with a suitable adhesive such as LensBond F65 (Summers Optical). This is done carefully after detector chips have been mounted and the wire leads have been attached. Black epoxy (EpoTek 320NC, Epoxy Technology) is painted on to mask any detector areas not covered by the filters, if any.

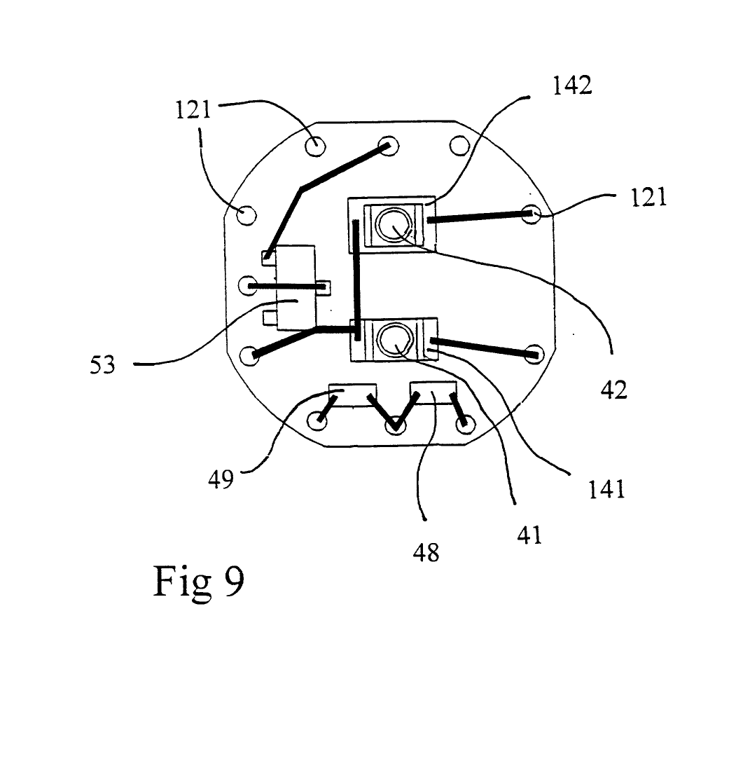

FIG. 9 shows the bottom circuit board with pads 141 and 142 for the LEDs 41 and 42, SOT-23 photodetector 53, feedback resistor 48, and feedback capacitor 49. Holes 121 are shown for the mounting pins along with circuit trace designations.

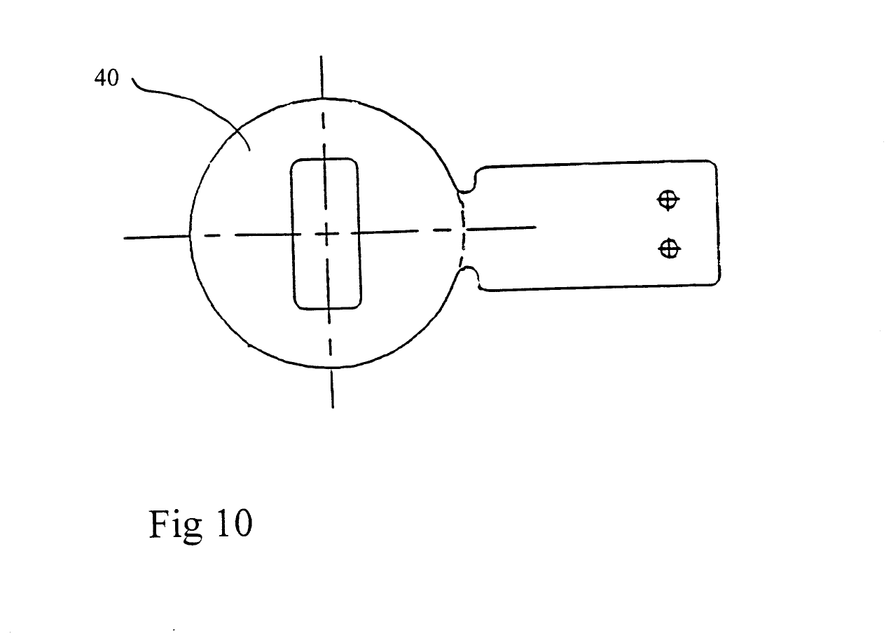

FIG. 10 is a top view of the heater 40 of the TO-8 can showing the slot dimensions. The heater used is of the thin foil type (Thermofoil by Minco) to minimize optical distances and thermal mass. The heating element is preferably of the high thermal coefficient of resistance type (Heaterstat by Minco) so the element can also be used as a temperature sensor. This reduces cost and reduces heater film thickness. The foil pattern is determined by the manufacturer to obtain a uniform thermal distribution.

Heat shrinking, chip bonding, soldering and associated cleaning operations are all performed prior to attachment of the optical filters. This is done to avoid excessive heat exposure, solvent or fume exposures, dust or debris contamination, or possible scratches to the filters. After attachment of the filters any remaining dust and debris are removed with an air spray cleaning. The TO-8 cans are then conditioned to remove moisture and sealed in accordance with normal TO can hermetic sealing practices.

Each sensor, whether for Oxygen, Carbon Dioxide, another analyte or combinations thereof are fabricated and assembled in a similar manner. The analyte sensitive target membrane, LEDs, and filters will vary depending on the analyte to be measured or the concentration range of the analyte.

### Target Layer Fabrication

In the case of PO₂ measurement, an oxygen sensitive organometallic fluorophor is immobilized or dispersed in a gas permeable polymer such as Polysiloxane. The fluorophor has an emission intensity which is inversely proportional to the Oxygen concentration in accordance with the Stern-Volmer relationship. This relationship is described by the equation:

I₀/I=1+k_{q}*t₀[Q] (1)

where I is the intensity, I₀ is the intensity without quenching, k_{q} is the rate constant of the quenching reaction, t₀ is the life of the emission in the absence of quencher, and [Q] is the concentration of quencher. t₀ and k_{q} are constants which vary widely among fluorophors. I₀ is a constant which must be calibrated for a given optical system with sensor.

This equation (1) is typically further refined to accommodate certain offsets in the system, temperature coefficients for the various constants, variations in the source light intensity, and other sources of non-linearity.

Quenchable fluorophors may be covalently bonded to a polymer matrix from which the dye is not leachable. They may also be adsorbed onto silica gels or dispersed from solvent in polymer where leaching is not a consideration. Oxygen quenchable fluorophors currently in use typically absorb light in the blue region of the optical spectrum and emit at longer wavelengths, typically in the green or red region of the spectrum.

Dynamic quenching of the fluorescence of certain organometallics by Oxygen in silicone is well known. Examples of such materials are Tris(4,7-Diphenyl-1,10-phenanthroline) Ruthenium (II) Chloride Hydrate (Ru-dpp), Tris(1,10-phenanthroline)Ruthenium (II) chloride Hydrate, and Tris(2,2′-bipyridine) Ruthenium (II) Chloride, to mention only a few. In the preferred embodiment of this invention Ru-dpp is used.

### EXAMPLE 1

By way of example, a 1 g of silica gel (Aldrich 288519) is stirred into 10 ml of a 2.5 mM solution of Ru-dpp (Oryza) in 0.025 Molar pH 6.838 phosphate buffer (RNA Medical) for one hour, then filtered. The filtrate is vacuum dried at 100 C. for 2 hours, then dispersed 5% w/w into a two part vinyl addition cure silicone (OE41, Gelest). The silicone is soaked into an open weave polyester filter medium (Reemay 2011) and oven cured at 60 C. for 2 hours.

The measurement of carbon dioxide is accomplished by correlating the pH change of a buffer to changes in carbon dioxide concentration equilibrated with the buffer. The carbon dioxide chemistry, the pH sensitive dye and a buffer, are contained in a silicone based hydrophobic polymer. Overall, the system is similar to what is known as a Severinghaus cell in which carbon dioxide diffuses through a membrane, forming carbonic acid in a buffered solution resulting in a pH change in the solution.

In a Severinghaus cell, the pH change is measured by a pH electrode system as a measurement of the partial pressure of carbon dioxide. In the carbon dioxide optical chemical sensor in accordance with this invention, a pH dye is used with the buffered solution. The dye is dissolved in the buffered solution and contained within a membrane. As increasing concentrations of carbon dioxide diffuse through the membrane the decrease in the pH of the buffer causes a change in the fluorescence of the dye. As the carbon dioxide level decreases, the process reverses and the pH increases, causing an inverse change in the fluorescence spectrum of the dye. Representative methods for computing the concentration of carbon dioxide in a medium by this technique are described in Lubbers et al U.S. Pat. No. Re. 31,879 and Heitzmann U.S. Pat. No. 4,557,900.

### EXAMPLE 2

A dye solution is prepared consisting of 6.5 mM 8-Hydroxy 1,3,6 pyrene trisulfonic acid (Kodak), 7 mM NaHCO₃ (Aldrich Chemical), 70 mM NaCl (Aldrich Chemical), and 10% w/w 100 k MW Polyethylene Oxide (Polysciences) in purified water. This solution is mixed until dissolved.

The target film is prepared by mixing 20% w/w of the dye solution into a two part vinyl addition cure silicone (OE41, Gelest). This material is emulsified with a Virtis Hand-Held Homogenizer, vacuum degassed for 3 minutes, then soaked into polyester filter discs (Reemay 2011). The discs are oven cured at 60 C. overnight.

Re-hydrate in clean warm water for 24 hours prior to testing.

### EXAMPLE 3

The dye mixture from Example 1 is loaded into a micro pipette and applied as an approximately 0.7 ul drop to a Whatman 54 filter disc. In a similar manner the emulsified and vacuum degassed material described in Example 2 is desiccated, loaded into a micro pipette, and applied as an approximately 0.7 ul drop adjacent to the earlier drop. The drops are spaced approximately 0.16″ apart with each spot wetting an area approximately 0.125″ in diameter. The disc is oven cured at 60 C. for 2 hours. The remainder of the filter is then wetted with additional silicone (OE41) to which 5% white pigment (United Chemical PSP051) and 5% Polyethylene Oxide 100 k MW (hydrated) has been added and cured.

The filter disc is then cut and mounted to a sensor cap with optically clear double coated adhesive. The disc is positioned such that each target film is located directly above its corresponding LED.

What is claimed is:

1. An optically based transcutaneous blood gas sensor comprised of a reusable portion and a disposable portion wherein the reusable portion consists of a windowed, hermetically sealed enclosure containing environmentally sensitive opto-electronic components, said enclosure being connected to an external monitor, and wherein the disposable portion has means of attachment to the reusable portion and carries fluorophores sensitive to the effects of O₂ and CO₂.

2. The apparatus of claim 1 wherein the reusable portion of the sensor contains LED light sources with peak wavelengths of 430 nanometers and 470 nanometers.

3. The apparatus of claim 1 wherein a heater and temperature sensing means are located in the outer surface of the reusable portion where it interfaces to the disposable portion and incorporates a clear area to allow for the passage of light.

4. The system of claim 1 wherein the analyte to be monitored is carbon dioxide and the fluorophor of the disposable portion of the sensor is 8-Hydroxy 1,3,6 pyrene trisulfonic acid.

5. The system of claim 1 wherein the analyte to be monitored is oxygen and the fluorophor of the disposable portion is Tris(4,7-Diphenyl-1,10-phenanthroline) Ruthenium (II).

6. The system of claim 1 wherein the target layer of the disposable portion of the sensor incorporates hydrophilic material allowing for the retention of water.

7. The system of claim 1 wherein a separate receptacle and support means for the sensor head has an outer sticky layer for attaching the complete sensor unit to the skin.

8. The apparatus of claim 1 wherein the light emitting diodes of the reusable portion are located on an inner substrate, and emission detecting photodetectors are located on a separate outer substrate, with the outer substrate containing openings for the outward passage of excitation light.

9. An optical based transcutaneous blood gas sensor comprising: a reusable sensor head having an enclosure with a cavity formed therein; a light source carried within said cavity; photodetector means carried within said cavity; a window means for sealing said light source and said photodetector means within said cavity; blood analyte fluorescent targets carried by a disposable cap for the sensor head; and support means for holding said fluorescent targets and said reusable sensor head adjacent the skin of a person, whereby said fluorescent targets are illuminated by said light source and produces an amount of fluorescence as a function of said blood analyte concentration, and said fluorescence is sensed by said photodetector means.

10. The apparatus of claim 9 wherein said fluorescent targets are carried by a disposable cap for the sensor head and wherein said support means comprises a separate receptacle for detachably carrying said sensor head.

11. The apparatus of claim 9 wherein said blood gas analyte is oxygen and the fluorescent target is Tris(4,7-Diphenyl-1,10-phenanthroline) Rutherium (II).

12. The apparatus of claim 9 wherein said blood gas analyte is carbon dioxide and said fluorescent target is 8-Hydroxy 1,3,6 pyrene trisulfonic acid.

13. The apparatus of claim 10 wherein said receptacle carries an adhesive layer for attaching the receptacle to the skin.

14. An optical based transcutaneous blood gas sensor comprising:
a reusable, hermetically sealed sensor head having an enclosure with a cavity formed therein,
a light source carried within said cavity,
photodetector means carried within said cavity,
a window means for sealing said light source and said photodetector means within said cavity,
a blood analyte fluorescent target, and
a disposable support means for detachably holding said fluorescent target adjacent said window means and for holding said fluorescent target and said reusable sensor head adjacent the skin of a person, whereby said fluorescent target is illuminated by said light source and produces an amount of fluorescence as a function of said blood analyte concentration, and said fluorescence is sensed by said photodetector means.

15. The apparatus of claim 14 further comprising a disposable cap for said reusable sensor head, and means for detachably connecting said disposable cap to said reusable sensor head.

16. The apparatus of claim 14 wherein said support means comprises a receptacle for detachably carrying said sensor head.

17. The apparatus of claim 14 wherein said blood gas analyte is oxygen and the fluorescent target is Tris(4,7-Diphenyl-1,10-phenanthroline) Rutherium (II).

18. The apparatus of claim 14 wherein said blood gas analyte is carbon dioxide and said fluorescent target is 8-Hydroxy 1,3,6 pyrene trisulfonic acid.

19. The apparatus of claim 16 wherein said receptacle carries an adhesive layer for attaching the receptacle and sensor head to the skin.
